(19) United States
(12) Patent Application Publication
(10) Pub. No.: US 2023/0266175 A1
(43) Pub. Date: Aug. 24, 2023

(54) MEASURING DEVICE

(71) Applicant: Nippon Telegraph and Telephone Corporation, Tokyo (JP)

(72) Inventors: Yujiro Tanaka, Tokyo (JP); Daichi Matsunaga, Tokyo (JP)

(21) Appl. No.: 18/006,949

(22) PCT Filed: Aug. 21, 2020
(86) PCT No.: PCT/JP2020/031652

§ 371 Date: Jan. 26, 2023

(51) Int. Cl. G01K 1/16; G01K 1/08

(52) U.S. Cl. CPC G01K 1/16; G01K 1/08; A61B 5/01

(57) ABSTRACT

A measuring device includes a first cover member having a measuring instrument, a second cover member configured to form an air layer between the second cover member and the first cover member, and a third cover member which transports heat flux from an object to be measured outside the first cover member to an upper part of the second cover member and a member having thermal conductivity and having a shape in which the member surrounds a side surface of the first member which are provided between the first cover member and the second cover member.

### CROSS-REFERENCE TO RELATED APPLICATIONS

This patent application is a national phase filing under section 371 of PCT application no. PCT/JP2020/031652, filed on Aug. 21, 2020, which application is incorporated herein by reference in its entirety.

### TECHNICAL FIELD

The present invention relates to a measuring device which measures a temperature of a deep portion of an object to be measured such as a living body.

### BACKGROUND

A technique for non-invasively measuring a body temperature of a deep portion of a living body is known in the related art. For example, Japanese Patent Application Publication No. 2020-003291 (“PTL 1”) discloses a technique of estimating a body temperature of a deep portion of a living body by assuming a pseudo one-dimensional model of a living body B, a measuring instrument 50 including a temperature sensor and a heat flux sensor, and outside air.

In the technique disclosed in PTL 1, the body temperature of a deep portion of a living body is estimated by assuming the one-dimensional model of biological heat transfer shown in FIG. 11. Tair is a temperature of the outside air, Tbody is a body temperature of a deep portion of the living body B, Hsignal is a temperature of the outside air, Rbody is a heat resistance of the living body B, Rair is a heat resistance when the heat flux Hsignal moves to the outside air, Tskin is a temperature of a contact point between a temperature sensor disposed on the skin SK and the skin SK of the living body B, and Tt is a temperature at a position in which the temperature sensor is disposed on an upper portion.

In PTL 1, the body temperature of a deep portion of a living body is estimated from the following relational Expression (1):

Expression (1):

Body temperature of deep portion (Tbody)=temperature of contact point between temperature sensor and skin (Tskin)+proportionality coefficient (Rsensor)×heat flux flowing into temperature sensor (Hsignal) (1)

Since the proportionality coefficient (Rsensor) can be obtained by providing a rectal temperature and an eardrum temperature measured by using a sensor such as another temperature sensor as the body temperature of the deep portion (Tbody), the body temperature of the deep portion of the living body can be estimated by measuring the heat flux (Hsignal) flowing into the temperature sensor.

### CITATION LIST

### Patent Literature

[PTL 1] Japanese Patent Application Publication No. 2020-003291.

### SUMMARY

### Technical Problem

However, when a one-dimensional model is assumed as a heat transfer model of a living body as in PTL 1, if heat flows from outside air to a sensor due to the generation of wind or the like, as shown in FIG. 12, a part of the heat flux Hsignal which originally would have flowed into the sensor is diverted from the sensor.

This is shown as in FIG. 13 illustrating a heat equivalent circuit thereof. Rbody is the thermal resistance of a living body, RLeak is the biothermal resistance when heat flows to the outside air due to wind or the like and is diverted from the original heat flow, and HLeak is the leaking heat flux. Rair and R′air are thermal resistances when Hsignal and HLeak move to the outside air, respectively. When the thermal resistance between the sensor and the outside air changes due to wind and a heat flux HLeak which leaks away from the sensor is generated, the heat flux Hsignal originally measured is reduced by that amount to become H′signal. Here, an influence of the HLeak on Hsignal is evaluated using a ratio Leak Ratio of HLeak to Hsignal. The ratio Leak Ratio is represented by |HLeak|/Hsignal.

Therefore, when wind or the like is generated, the ratio Leak Ratio becomes large, and the one-dimensional model is no longer established in Hsignal, and in a technique of measuring a body temperature of a deep portion in the related art, there is a problem that the body temperature of a deep portion is erroneously measured when wind or the like is generated around the sensor.

Embodiments of the present invention were made to solve the above-mentioned problem, and an object of embodiments of the present invention is to provide a measuring device capable of preventing a change in thermal resistance between a sensor and outside air and accurately measuring a body temperature of a deep portion even when wind or the like is generated around the sensor.

### Solution to Problem

In order to solve the above problem, embodiments of the present invention relate to a measuring device which includes: a measuring instrument configured to measure a heat flux transported from an object to be measured; a first member having a hollow structure and having the measuring instrument therein; a second member having a hollow structure and configured to cover the first member to form an air layer between the first member and the second member; a third member disposed between the first member and the second member and configured to transport a heat flux from the object to be measured outside the first member to an upper part of the second member; and a fourth member having thermal conductivity and having a shape in which the fourth member surrounds at least a side surface of the first member.

### Advantageous Effects of Embodiments of the Invention

According to embodiments of the present invention, the first member having the measuring instrument and the second member which forms the air layer between the second member and the first member are provided, and the third member which transports a heat flux from the object to be measured outside the first member to the upper part of the second member between the first member and the second member and the fourth member having thermal conductivity and having a shape in which the fourth member surrounds at least the side surface of the first member are further provided. Thus, it is possible to provide a measuring device capable of preventing change in thermal resistance between the sensor and the outside air and accurately measuring the body temperature of a deep portion even when wind is generated around the sensor.

### BRIEF DESCRIPTION OF THE DRAWINGS

FIG. 1 is an example of a cross-sectional view of a measuring device according to an embodiment of the present invention.

FIG. 2 is another example of a cross-sectional view of a measuring device according to an embodiment of the present invention.

FIG. 3 is a diagram showing an example of second and third members of a measuring device according to an embodiment of the present invention.

FIG. 4 is a diagram showing an example of a third member of a measuring device according to an embodiment of the present invention.

FIG. 5A is a diagram showing another example of a cross-sectional view of a measuring device according to an embodiment of the present invention.

FIG. 5B is a diagram showing another example of a cross-sectional view of a measuring device according to an embodiment of the present invention.

FIG. 5C is a diagram showing another example of a cross-sectional view of a measuring device according to an embodiment of the present invention.

FIG. 5D is a diagram showing another example of a cross-sectional view of a measuring device according to an embodiment of the present invention.

FIG. 6 is a diagram showing an example of a cross-sectional view of a third member and a fourth member of a measuring device according to an embodiment of the present invention.

FIG. 7 is a diagram showing a temperature field and a heat flux in the vicinity of a measuring device according to an embodiment of the present invention.

FIG. 8 is a diagram showing a heat equivalent circuit of FIG. 7.

FIG. 9 is a measurement result of a measurement error of a temperature of a deep portion according to an embodiment of the present invention.

FIG. 10 is an example of a block diagram of a measuring device according to an embodiment of the present invention.

FIG. 11 is a heat equivalent circuit for estimating a temperature of a deep portion using a heat flux.

FIG. 12 is a diagram for explaining a leak heat flux when estimating a temperature of a deep portion using a heat flux.

FIG. 13 is a heat equivalent circuit diagram when a leak heat flux occurs.

### DETAILED DESCRIPTION OF ILLUSTRATIVE EMBODIMENTS

Preferred embodiments of the present invention will be described below. In the following embodiments, the object to be measured is a living body and a surface to be measured on which the measuring device is disposed is a surface of a skin of a living body to be measured.

### Outline of Embodiments of the Present Invention

A measuring device of embodiments of the present invention includes: a first member having a hollow structure and having a measuring instrument for measuring a heat flux therein and a second member having a hollow structure and configured to form an air layer between the first member and the second member which are provided therein; and a third member configured to transport a heat flux from an object to be measured outside the first member to an upper part of the second member between the first member and the second member and a fourth member having thermal conductivity and having a shape in which the fourth member surrounds at least a side surface of the first member which are further provided.

A measuring device of embodiments of the present invention includes in addition to a first member having a measuring instrument for measuring a heat flux and a second member which forms an air layer between the first member and a third member, the third member which transports a heat flux from an object to be measured to an upper part of the second member, and a fourth member which has thermal conductivity and surrounds at least a side surface of the first member so that the temperature of the upper part of the measuring instrument can be raised and the temperature around the measuring instrument can be kept symmetrical. Therefore, it is possible to prevent a change in thermal resistance between the measuring instrument and the outside air, prevent a leak heat flux which causes a measurement error in the temperature of a deep portion, and reduce the ratio Leak Ratio even when wind is generated around the measuring device. A specific configuration of the measuring device of the present embodiment will be described below.

### Configuration of Measuring Device

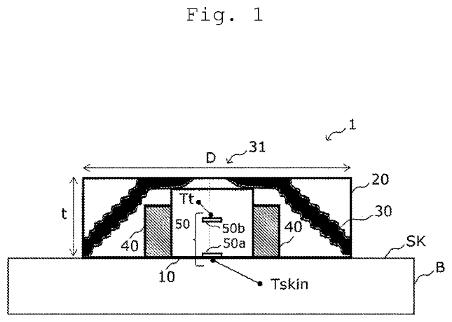

FIG. 1 shows an example of a cross-sectional view of a measuring device according to an embodiment of the present invention. FIG. 1 shows an example of a configuration of a first member 10 having a measuring instrument 50 therein, a second member 20 covering the first member 10, a third member 30 disposed in a space between the first member 10 and the second member 20, and a fourth member 40 surrounding a side surface of the first member 10. The measuring instrument 50 disposed inside the first member 10 includes a sensor for measuring a heat flux transported from a living body B. Note that, although not shown in FIG. 1, the measuring device 1 includes an arithmetic circuit for estimating a temperature of a deep portion of the living body B in addition to the configuration of the measuring device 1 shown in FIG. 1.

The measuring device 1 of FIG. 1 includes the first member 10 having a hollow structure in which the first member 10 holds the measuring instrument 50 therein, the second member 20 having a hollow structure in which the second member 20 covers the first member 10 and forms an air layer between the first member 10 and the second member 20, the third member having a truncated cone shape and a hollow shell structure in which the third member 30 is disposed in the space between the second member 20 and the first member 10, and the fourth member 40 which has a ring shape and surrounds the first member 10.

In the configuration example shown in FIG. 1, the truncated cone-shaped upper surface portion of the third member 30 is in contact with the upper surface portion of the second member 20 from the inside of the second member 20. Furthermore, the truncated cone-shaped third member 30 has a hole 31 which passes through the third member 30 on the upper surface portion thereof. The upper surface portion of the third member 30 comes into contact with the upper surface portion of the second member 20 so that a heat flux from the living body B outside the first member 10 is transported to the upper surface portion of the second member 20.

The fourth member 40 is made of a material having thermal conductivity, surrounds a side surface of the first member having a hollow structure in which the first member holds the measuring instrument 50 therein and is configured to keep a temperature around the measuring instrument 50 symmetrical. A shape of the fourth member 40 is changed in accordance with a shape of the first member 10. For example, if the first member 10 has a cylindrical shape, the fourth member 40 surrounding the first member 10 is a circular ring.

The measuring instrument 50 disposed inside the first member 10 includes a temperature sensor 50a (first temperature sensor) configured to measure a temperature of a skin SK which is a surface to be measured and a temperature sensor 50b (second temperature sensor) disposed at a position just above the temperature sensor 50a to face the temperature sensor 50a. In the configuration example of FIG. 1, a heat flux is measured by using a temperature difference between a temperature Tskin measured by the temperature sensor 50a and a temperature Tt measured by the temperature sensor 50b.

The first member 10 has a hollow structure and the inside thereof is filled with air. It is desirable that the second member 20 be filled with a material having a large thermal resistance and a cavity such as air can be used.

A material (about 0.1 mm) having a small thermal resistance and a thin thickness is preferable for the first member 10 and the second member 20 and polyethylene terephthalate (PET) or the like can be used. It is preferable that a material have a high thermal conductivity to efficiently transport a heat flux as a material of the truncated cone-shaped third member 30 having the hollow shell structure. For example, the third member 30 may be formed of a thin film made of aluminum or the like.

A material having a high thermal conductivity is preferable as a material of the fourth member 40, as in the third member 30, to keep a temperature around the measuring instrument 50 symmetrical. For example, aluminum or the like can be used for the material.

The first member 10 is disposed on the skin SK of the living body B which is a surface to be measured. The first member 10 has a hollow structure in which the first member 10 is formed of a thin film and can have, for example, a cylindrical outer shape. The second member 20 covers the first member 10 and is disposed on the skin SK of the living body B which is a surface to be measured and forms an air layer between the first member 10 and the second member 20. The second member 20 has a hollow structure in which the second member 20 is formed of a thin film like the first member 10 and can have a cylindrical outer shape. Note that the outer shapes of the first member 10 and the second member 20 are not limited to cylindrical shapes, and may be, for example, a rectangular parallelepiped shape having a hollow structure.

A diameter D of a cylindrical shape of the first member 10 and a diameter D of a cylindrical shape of the second member 20 can be set to, for example, 20 mm and 30 mm, respectively. A height t of the second member 20 with respect to the skin SK which is the surface to be measured can be set to, for example, about 6 mm. A height of the first member 10 with respect to the skin SK which is the surface to be measured can be set to, for example, about 3 mm.

In this way, a configuration is provided in which an air layer formed by the first member 10 and an air layer between the first member 10 and the second member 20 outside the first member 10 are formed and air in each of the first member 10 and the second member 20 does not move.

Furthermore, a configuration is provided in which the third member 30 is disposed between the first member 10 and the second member 20 and the upper surface portion of the third member 30 comes into contact with the upper surface portion of the second member 20 so that the heat flux from the living body B is transported to the upper part of the second member 20 outside the first member 10. In the example shown in FIG. 1, since the truncated cone-shaped third member 30 has a hole 31 which passes through the third member 30 on the upper surface portion thereof, the upper surface portion of the second member 20 is in contact with the peripheral portion of the hole 31 of the upper surface portion.

The fourth member 40 is constituted to surround a side surface around the first member 10 having a hollow structure in which the fourth member 40 holds the measuring instrument 50 therein. When the fourth member which surrounds the side surface around the first member 10 is provided in addition to the third member 30, since the temperature of the upper part of the measuring instrument is raised, the temperature around the measuring instrument is kept symmetrical, and it is possible to prevent a change in thermal resistance between the measuring instrument and the outside air and to prevent a leak heat flux which causes a measurement error in the temperature of the deep portion even when wind is generated around the measuring instrument.

### Configuration of Sensors in the Measuring Instrument

The temperature sensor 50a is disposed on an inner surface of a bottom surface portion in which the cylindrical first member 10 is in contact with the skin SK which is the surface to be measured. The temperature sensor 50b is disposed on the inner surface of the upper surface portion of the first member 10 to face the temperature sensor 50a at a position just above the temperature sensor 50a. In the configuration example of FIG. 1, a heat flux H′signal is measured using a temperature difference between the pair of temperature sensors 50a and 50b.

In FIG. 1, the temperature sensor 50a is disposed to be in contact with the surface of the skin SK of the living body B which is the surface to be measured and measures the temperature Tskin (temperature of the surface to be measured) which is a temperature of a contact point with the living body B. The temperature sensor 50b measures a temperature Tt at a disposition position of an inner surface of the first member 10. For example, thermistors, thermocouples, platinum resistors, IC temperature sensors, or the like can be used as the temperature sensors 50a and 50b.

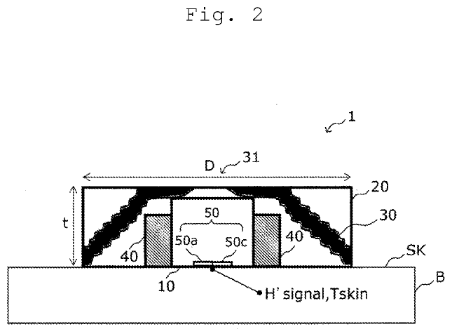

Although a configuration in which the heat flux H′signal is measured using the pair of temperature sensors 50a and 50b is provided in the configuration example of FIG. 1, the temperature sensor 50a may be used for measuring the temperature Tskin of the surface to be measured and a heat flux sensor 50c may be used for measuring the heat flux H′signal as shown in FIG. 2.

In FIG. 2, the heat flux sensor 50c is a sensor which detects heat transfer per unit area for a unit time and measures the heat flux H′signal [W/m2] flowing from the living body B to the heat flux sensor 50c. For the heat flux sensor 50c, for example, a laminated structure, a planar expansion type operation type thermopile, or the like can be used. The heat flux sensor 50c is disposed to be in contact with the surface of the skin SK of the living body B which is the surface to be measured.

In FIG. 2, the temperature sensor 50a is disposed to be in contact with the skin SK which is the surface to be measured as in FIG. 1 and measures the skin temperature Tskin which is the temperature of the contact point with the living body B. The temperature sensor 50a is disposed adjacent to the heat flux sensor 50c along the surface to be measured.

### Configuration Example of Third Member

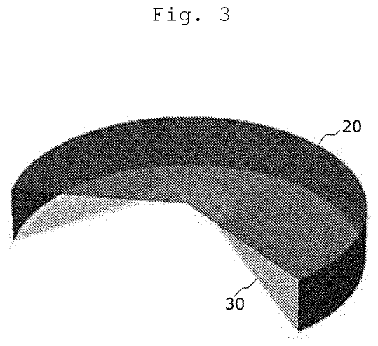

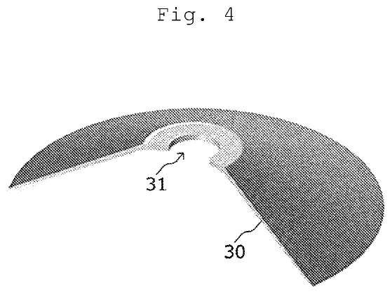

FIGS. 3 and 4 show a configuration example of the third member 30. In FIGS. 3 and 4, a configuration in which the cylindrical second member 20 is disposed to cover the truncated cone-shaped third member 30 and an upper surface portion of the truncated cone-shaped third member 30 is in contact with an upper surface portion of the cylindrical second member 20 is provided. Furthermore, the truncated cone-shaped third member 30 has the circular hole 31 which passes through the third member 30 on the upper surface portion thereof.

The third member 30 is a member which is disposed in the space between the first member 10 and the second member 20, transports the heat flux from the object to be measured to the upper surface portion of the second member outside the first member so that the temperature of the upper surface portion of the second member, that is, the temperature of the upper part of the measuring instrument 50, is raised, and functions to prevent the leak heat flux HLeak and lower the ratio Leak Ratio. As the configuration of the third member 30, configurations having various shapes can be adopted as long as the configuration can exhibit this function.

For example, when the third member is disposed between the first member and the second member having a cylindrical shape, the third member can be configured to have a circular thrust shape cone. The heat flux from the object to be measured can be transported to the upper surface portion of the second member outside the first member without affecting the heat flux flowing into the measuring instrument 50 by forming the third member in a cone shape. The third member can also be configured to have a truncated cone shape as shown in FIGS. 3 and 4.

Furthermore, the configuration of the third member 30 is not limited to a cone shape or a truncated cone shape and can have other cone shapes. For example, when the second member 20 has a rectangular parallelepiped shape, the third member 30 can have a pyramid shape or a truncated pyramidal shape corresponding to the rectangular parallelepiped shape. More heat flux can be transported to the second member without affecting the heat flux flowing into the measuring instrument 50 and the effect of temperature rise can be enhanced by forming the third member in a truncated pyramidal shape.

Furthermore, as illustrated in FIG. 1 to FIG. 4, the truncated cone-shaped third member 30 may be configured to include the circular hole 31 which passes through the third member 30 on the upper surface portion thereof. It is possible to adjust a depth to be measured at the time of measuring a temperature of a deep portion of the living body B by appropriately adjusting a size of the circular hole 31.

### Configuration Example of Fourth Member

The fourth member 40 is constituted to surround the peripheral side surface of the first member 10 having a hollow structure in which the fourth member 40 holds the measuring instrument 50 therein. When the fourth member which surrounds the side surface around the first member is provided in addition to the third member 30, it is possible to prevent a change in thermal resistance between the measuring instrument 50 and the outside air and to prevent the leak heat flux which causes a measurement error of a temperature of a deep portion even when the temperature of the upper part of the measuring instrument is raised, the temperature around the measuring instrument is kept symmetrical, and wind is generated around the measuring instrument 50.

As the configuration of the fourth member 40, configurations having various shapes can be adopted as long as the shape can exhibit this function.

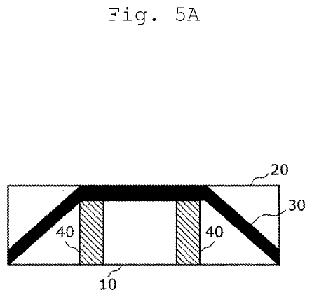

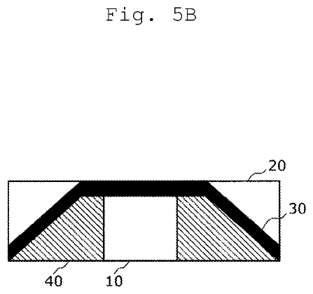

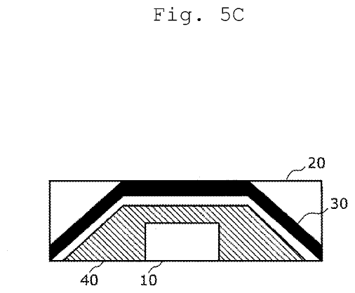

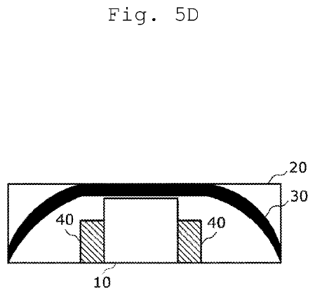

For example, as shown in FIG. 5A, a height of the fourth member 40 and a height of the first member 10 may be configured to be set to substantially the same degree and the upper part of the fourth member 40 may come into contact with the upper part of the third member 30 from the inner surface of the third member 30, or as shown in FIG. 5B, the space between the third member 30 and the first member 10 may be configured to be filled with the fourth member 40, or as shown in FIG. 5C, the third member 30 may be configured to cover the upper surface and the side surface of the first member 10. Furthermore, as shown in FIG. 5D, the fourth member 40 and the first member 10 may be disposed inside the third member 30 having a dome shape or a spherical shape.

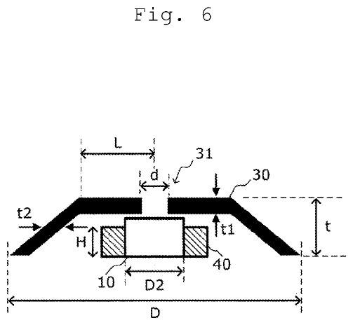

FIG. 6 is an example of a cross-sectional view of the truncated cone-shaped third member 30 and the fourth member 40 having the hole 31 in the upper surface portion. As an example of the size of the third member 30 in the embodiment, when a diameter D of the second member 20 is 30 mm and a height t is 5 mm, a radius L of the upper surface portion is 3 mm to 6 mm, and a diameter d of the hole 31 is about 1 mm to 3 mm. The hole 31 may be omitted and the diameter d=0 mm may be used depending on the size of each member. Furthermore, it is preferable that thicknesses t1 and t2 of the third member 30 be, for example, about 0.3 mm to 1 mm. It is preferable that the third member 30 and the first member 10 have substantially the same height.

It is preferable that a height H of the fourth member 40 be about 2 mm, an inner diameter D2 be about 3 to 6 mm, and a thickness of a ring in a ring structure be about 1 to 4 mm. It is preferable that an outer shape of the fourth member 40 surrounding the first member lo be the same as that of the first member 10. For example, if the first member 10 has a cylindrical shape, the fourth member 40 surrounding the first member 10 is a circular ring.

### Temperature Field and Heat Flux in an Embodiment

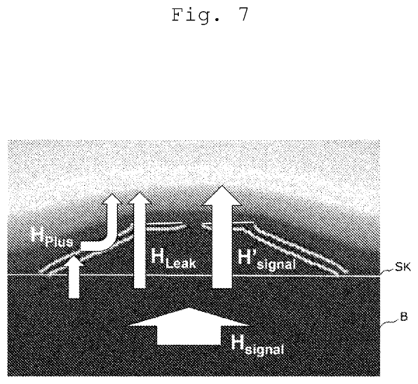

FIG. 7 is a diagram showing a temperature field and a heat flux in the vicinity of the measuring device. A heat flux Hplus is a heat flux which is transported from the living body B to the vicinity of a central part of the upper part of the second member 20 outside the first member 10 through the truncated cone-shaped third member 30 and the fourth member 40 surrounding the first member 10.

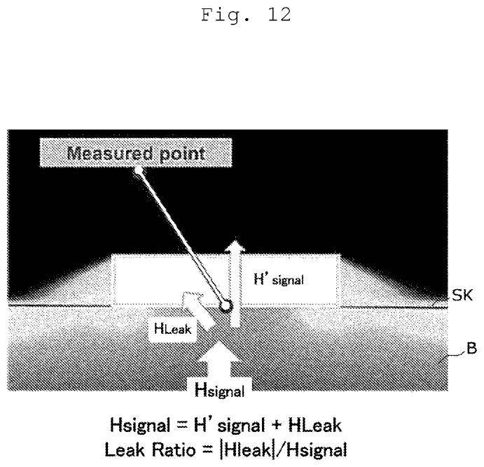

In FIG. 7, Hsignal is a heat flux transported from a deep portion of the living body B, H′signal is a heat flux separated from the Hsignal and flowing into a central temperature sensor, and HLeak is a leak heat flux separated from the Hsignal, deviating from the measuring instrument 50, and escaping to the outside. As in FIG. 12, in this case, the ratio Leak Ratio of HLeak to Hsignal is represented by |HLeak|/Hsignal.

### Heat Equivalent Circuit of an Embodiment

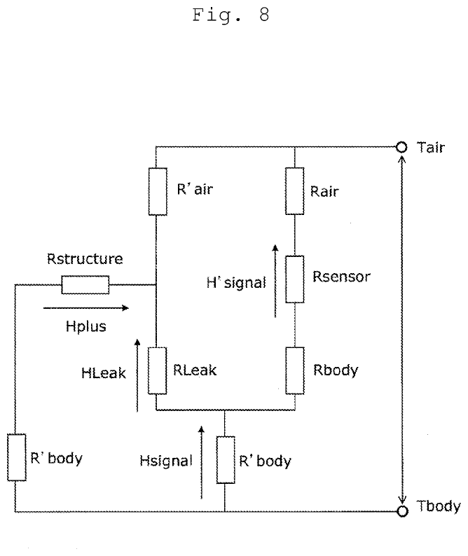

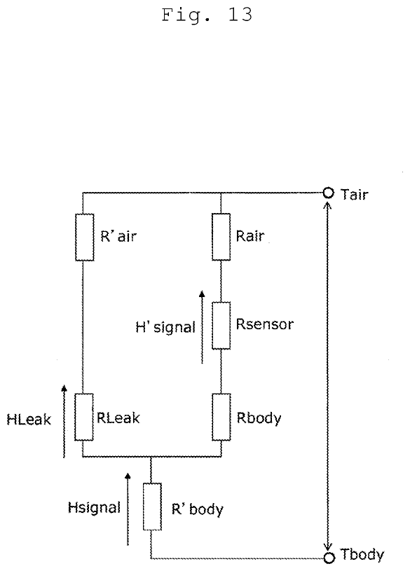

FIG. 8 shows the heat equivalent circuit of FIG. 7. Rstructure is thermal resistance of the truncated cone-shaped third member 30 and the fourth member 40, and R′body is a thermal resistance when heat is transferred from the deep portion to the truncated cone-shaped third member 30, and as described with reference to FIG. 13, HLeak is the thermal resistance when moving to the outside air. Rair and R′air are thermal resistance when heat is transported to the outside air through the measuring instrument 50 and thermal resistance when the heat is transported to the outside air deviating from the measuring instrument 50, respectively.

Here, when the truncated cone-shaped third member 30 is sufficiently large, an end part of a bottom surface of the truncated cone-shaped third member 30 is located at a position in which the end part is sufficiently separated from the measuring instrument 50. Thus, the heat flux from the living body B is collected by the third member 30 and transported to the upper surface portion of the second member 20 outside the first member 10. Furthermore, the heat flux collected by the fourth member 40 is also transported to the upper surface portion of the second member 20.

The heat flux Hplus collected and transported using the truncated cone-shaped third member 30 and the fourth member 40 increases the temperature of the upper surface portion of the second member 20 without affecting the Hsignal, and as a result, the temperature outside the measuring instrument 50 can be raised. In the heat equivalent circuit shown in FIG. 7, when the heat flux Hplus flows into the R′air, the temperature outside the measuring instrument rises, and the leak heat flux HLeak which causes an error can be prevented and the effect of lowering the ratio Leak Ratio can be produced.

The truncated cone-shaped third member 30 is covered with the second member 20 and a distance from the outside air becomes smaller toward the vicinity of the central part in which the measuring instrument 50 is disposed and becomes almost zero near the central part in which the measuring instrument 50 is disposed. Thus, the closer to the vicinity of the center part, the more the suppression effect of the inflow of heat from the outside air to the sensor becomes larger, and the highest reduction effect of the ratio Leak Ratio can be obtained in the vicinity of the center part in which the measuring instrument 50 is disposed. As a result, a difference between the heat flux H′signal measured by the temperature sensor or the heat flux sensor and the Hsignal originally desired to be measured can be reduced and the measurement error can be reduced.

### Measurement Error Comparison Result

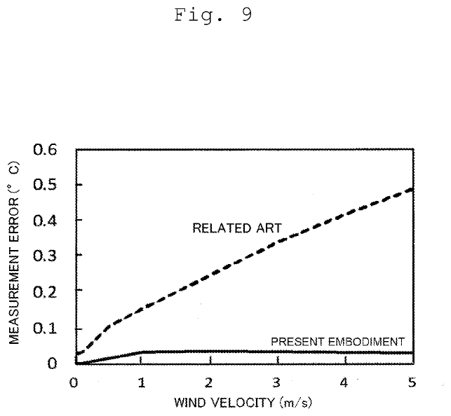

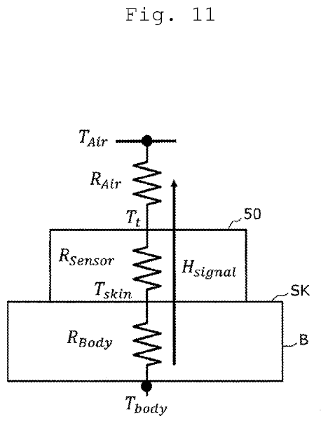

FIG. 9 shows measurement results of a measurement error of a temperature of a deep portion in the measuring device 1. FIG. 9 shows a relationship between a wind velocity and a measurement error when wind is applied to the measuring device 1. An embodiment of the present invention in the drawing is a measurement result in the configuration shown in FIG. 1 and the related art is a measurement result in the configuration shown in FIGS. 11 and 12. It is assumed that a maximum wind speed given to the measuring device 1 is 5 m/s and jogging is performed at about 18 km/h. According to the measuring device of the embodiment, it can be confirmed that the measurement error of the temperature of the deep portion can be minimized to 0.1° C. or less.

### Effect of Embodiments

According to the embodiments, the first member 10 having the measuring instrument for measuring heat flux and the second member 20 configured to form the air layer between the second member 20 and the first member 10 are provided, and the third member 30 which transports heat flux from the object to be measured outside the first member 10 and the fourth member 40 surrounding the first member 10 are further provided between the first member 10 and the second member 20. Thus, it is possible to prevent a change in thermal resistance between the sensor and the outside air, to prevent the leak heat flux which causes a measurement error, and to reduce a measurement error at the time of measuring a temperature of a deep portion by lowering the ratio Leak Ratio even when the heat flux transported to the upper surface portion of the second member 20 raises the temperature outside the measuring instrument 50, the temperature around the measuring instrument 50 is kept symmetrical, and wind is generated around the measuring device 1.

### Configuration Example of Measuring Device

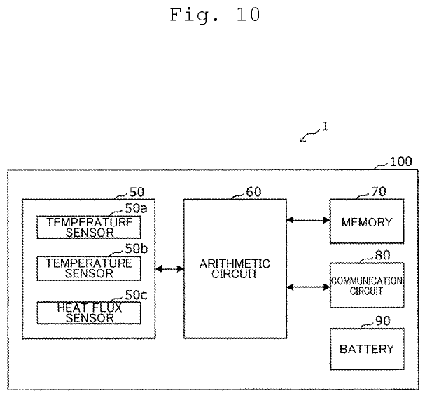

The configuration of the measuring device 1 according to an embodiment will be described with reference to FIG. 10. As shown in FIG. 10, the measuring device 1 includes the configuration of the measuring device 1 described with reference to FIG. 1, an arithmetic circuit 60 for estimating a body temperature of a deep portion, a memory 70, a communication circuit 80, and a battery 90.

The measuring device 1 includes, for example, the measuring instrument 50, the arithmetic circuit 60, the memory 70, the communication circuit 80 which functions as an I/F circuit with the outside, and the battery 90 which supplies electric power to the arithmetic circuit 60, the communication circuit 80, and the like on a sheet-shaped base material loft

In the configuration example of FIG. 1, the arithmetic circuit 60 calculates an estimated value of a body temperature TC of a deep portion from temperatures Tskin and Tt measured by the temperature sensors 50a and 50b provided in the measuring instrument 50 by using Expression (1).

In the configuration example of FIG. 2, the arithmetic circuit 60 calculates an estimated value of the body temperature TC of the deep portion by using Expression (1) from the heat flux Hsignal and the skin temperature Tskin measured by the heat flux sensor floc and the temperature sensor 50a provided in the measuring instrument 50.

The memory 70 stores information on a one-dimensional heat transfer model based on the foregoing Expression (1) and the estimation result of the body temperature of the deep portion. The memory 70 can be realized by a predetermined storage region in a rewritable nonvolatile storage device (for example, a flash memory) provided in the measurement system.

The communication circuit 80 outputs the time-series data of the body temperature TC of the deep portion of the living body B generated by the arithmetic circuit 60 to the outside. Such a communication circuit 80 becomes an output circuit to which a USB or other cable can be connected when data or the like is output by wire, but for example, a wireless communication circuit compliant with Bluetooth (registered trademark), Bluetooth Low Energy, or the like may be used.

The sheet-shaped base material 100 functions as a base which has the measuring device 1 including the measuring instrument 50, the arithmetic circuit 60, the memory 70, the communication circuit 80, and the battery 90 installed thereon and includes a wiring (not shown) for electrically connecting these elements. Assuming that the measuring device 1 is connected on the epidermis of a living body, it is preferable to use a deformable flexible substrate for the sheet-shaped base material 100.

Furthermore, the opening is provided in a part of the sheet-shaped base material 100 and the temperature sensor 50a included in the measuring instrument 50 and the heat flux sensor 50c are placed on the base material 100 to be in contact with the surface to be measured of the skin SK of the living body B through the opening.

Here, the measuring device 1 is realized using a computer. Specifically, the arithmetic circuit 60 is realized by performing various data processing in accordance with programs stored in a storage device such as a ROM, a RAM, and a flash memory including a memory 70 provided in the measuring device 1 by, for example, a processor such as a CPU or a DSP. The above program for operating the computer as the measuring device 1 can be recorded on a recording medium or provided through a network.

Note that, although the configuration of the measuring device 1 including the measuring instrument 50 described with reference to FIG. 1 is integrally configured with other configurations including the arithmetic circuit 60 in the measuring device 1 in FIG. 9, the configuration of FIG. 1 may be a configuration separated from the arithmetic circuit 60, the memory 70, the communication circuit 80, and the battery 90. For example, the configuration of the measuring device 1 and the other units such as the arithmetic circuit 60 and the like may be connected via a wiring (not shown).

### Modified Example of Embodiments

Although the measuring device of embodiments of the present invention have been described above, the present invention is not limited to the embodiments which have been described and various modifications which can be assumed by those skilled in the art can be made within the scope of the invention described in the claims.

### REFERENCE SIGNS LIST

1 Measuring device

10 First member

20 Second member

30 Third member

31 Hole

40 Fourth member

50 Measuring instrument

50a, 50b Temperature sensors

50c Heat flux sensor

60 Arithmetic circuit

70 Memory

80 Communication circuit

90 Battery

100 Base material

1.-8. (canceled)

9. A measuring device comprising:
a first member having a first hollow structure;
a measuring instrument in the first member, wherein the measuring instrument is configured to measure a heat flux transported from an object to be measured;
a second member having a second hollow structure, wherein the second member covers the first member to define an air layer between the second member and the first member;
a third member disposed between the first member and the second member, wherein the third member is configured to transport the heat flux from the object to be measured outside the first member to an upper part of the second member; and
a fourth member having thermal conductivity and having a shape in which the fourth member surrounds a side surface of the first member.

10. The measuring device according to claim 9, wherein:
the third member has a cone shape; and
an upper part of the third member having the cone shape contacts the upper part of the second member from an inner surface of the second member.

11. The measuring device according to claim 9, wherein:
the third member has a frustum shape; and
an upper surface portion of the third member having the frustum shape contacts the upper part of the second member from an inner surface of the second member.

12. The measuring device according to claim 9, wherein:
the second member has a cylindrical shape;
the third member has a truncated cone shape; and
an upper surface portion of the third member having the truncated cone shape contacts an upper surface portion of the second member having the cylindrical shape from an inner surface of the second member.

13. The measuring device according to claim 12, wherein the third member comprises a hole that passes through the third member on the upper surface portion of the third member having the truncated cone shape.

14. The measuring device according to claim 9, wherein the fourth member has a ring shape in which the fourth member surrounds the side surface of the first member.

15. The measuring device according to claim 9, wherein an upper part of the fourth member contacts the third member from an inner surface of the third member.

16. The measuring device according to claim 9, wherein the fourth member covers an upper surface and the side surface of the first member.

17. A method of forming a measuring device, the method comprising:
providing a first member having a first hollow structure;
providing a measuring instrument within the first member, wherein the measuring instrument measures a heat flux transported from an object to be measured;
providing a second member having a second hollow structure, wherein the second member covers the first member to define an air layer between the second member and the first member;
providing a third member between the first member and the second member, wherein the third member transports the heat flux from the object to be measured outside the first member to an upper part of the second member; and
providing a fourth member having thermal conductivity and having a shape in which the fourth member surrounds a side surface of the first member.

18. The method according to claim 17, wherein:
the third member has a cone shape; and
an upper part of the third member having the cone shape contacts the upper part of the second member from an inner surface of the second member.

19. The method according to claim 17, wherein:
the third member has a frustum shape; and
an upper surface portion of the third member having the frustum shape contacts the upper part of the second member from an inner surface of the second member.

20. The method according to claim 17, wherein:
the second member has a cylindrical shape;
the third member has a truncated cone shape; and
an upper surface portion of the third member having the truncated cone shape contacts an upper surface portion of the second member having the cylindrical shape from an inner surface of the second member.

21. The method according to claim 20, wherein the third member comprises a hole that passes through the third member on the upper surface portion of the third member having the truncated cone shape.

22. The method according to claim 17, wherein the fourth member has a ring shape in which the fourth member surrounds the side surface of the first member.

23. The method according to claim 17, wherein an upper part of the fourth member contacts the third member from an inner surface of the third member.

24. The method according to claim 17, wherein the fourth member covers an upper surface and the side surface of the first member.
